(19) United States
(12) Patent Application Publication
(10) Pub. No.: US 2009/0197344 A1
(43) Pub. Date: Aug. 6, 2009

(54) METHOD FOR THE PREDICTION OF VASCULAR EVENTS AND THE DIAGNOSIS OF ACUTE CORONARY SYNDROME

(76) Inventors: Sylvie Villard-Saussine, Issy Les Moulineaux (FR); Estelle Le Moal, Puteaux (FR); Catherine French Larue, Vaucresson (FR); Isabelle Giuliani, Garches (FR)

Correspondence Address: JACOBSON HOLMAN PLLC, 400 SEVENTH STREET N.W., SUITE 600, WASHINGTON, DC, 20004, US

(21) Appl. No.: 12/309,870

(22) PCT Filed: Aug. 7, 2007
(86) PCT No.: PCT/IB2007/002266

§ 371 Date: Feb. 2, 2009

Related U.S. Application Data
(60) Provisional application No. 60/835,903, filed on Aug. 7, 2006.

(30) Foreign Application Priority Data
Aug. 7, 2006 (EP) 06291277.9

(51) Int. Cl. G01N 33/00; A61B 5/0452

(52) U.S. Cl. 436/86; 600/509

(57) ABSTRACT

The present invention relates to a method for the prognosis of a vascular event or the diagnosis of ACS in a patient suspected of being at risk for a vascular event or condition, said patient presenting:
no elevation of the ST segment as seen on an electrocardiogram, and/or
a normal level of at least one necrosis marker,
   wherein the presence and/or levels of at least two different biochemical markers are measured in a biological sample of said patient, whereby the probability that the patient will experience a vascular event or the vascular-related condition is deduced from the measured presence and/or levels of the biochemical markers.

The present application claims the benefit of prior U.S. provisional application 60/835,903 filed Aug. 8, 2006.

The present invention relates to a method for predicting the occurrence of vascular events in patients suspected of being at risk for such events.

Each year, millions of patients present to hospitals with symptoms, such as chest pain, indicative of acute coronary syndrome (ACS). ACS refers to a variety of clinical symptoms caused by acute myocardial ischemia. Identification of patients having ACS is of tremendous importance, due to their higher risk for cardiac death or ischemic complications.

The current protocols for assessing the status of patients suspected of ACS usually include performing an electrocardiogram (ECG) and determining the blood level of a necrosis marker, in particular a myocardial necrosis marker, commonly cardiac Troponin I or T (cTnI or cTnT) or creatine kinase (CK).

Briefly, as defined by Grech et al. (2003) BMJ 326:1259-1261, patients presenting with an acute chest pain and submitted to the above tests are classified as follows:
no ST segment elevation and marker of myocardial necrosis not elevated: unstable angina;
no ST segment elevation and elevated level of a marker of myocardial necrosis: non-ST myocardial infarction;
ST segment elevation and elevated level of a marker of myocardial necrosis: ST myocardial infarction.

Unstable angina, non-ST myocardial infarction, and ST myocardial infarction are all constitutive of an ACS.

Among the patients tested, those presenting no elevation of the ST segment on the electrocardiogram recording and normal levels of the necrosis markers, in particular the myocardial necrosis markers, are particularly difficult to deal with. On one hand, admission of these patients is most often unnecessary because of their limited risk of enduring adverse vascular events, with unnecessary admissions bearing high costs on health protection systems. On the other hand, not all of these patients are risk-free, notably because the presentation of an ACS is often atypical, and it is estimated that as much as 1-2% of patients who are sent home have an adverse vascular event afterwards, such as myocardial infarction (Apple et al. (2005) Clinical Chemistry 51:810-824).

As such, among patients suspected of being at risk for a vascular event and who present with no elevation of the ST segment and normal levels of a necrosis marker, in particular a myocardial necrosis marker, there is an important need for a method to help discriminate between risk-free patients and those effectively at risk of undergoing an adverse vascular event.

Therefore, it is an object of the present invention to provide a method for the prognosis of vascular events in patients who present no elevation of the ST segment and normal levels of a necrosis marker, in particular a myocardial necrosis marker.

It is another object of the invention to provide a reliable method for diagnosing ACS among these patients.

It is yet another object of the invention to provide a method for determining the condition of an individual suspected of having an ACS.

It is a further object of the invention to provide kits for implementing the above methods.

The present invention arises from the demonstration, by the Inventors, that the probability of future vascular events in patients suspected of being at risk for such events can be determined by:
measuring the presence and/or the level of at least two different biochemical markers selected from the group consisting of the following classes of markers:
   atherosclerotic plaque unstability markers,
   atherosclerotic plaque disruption markers,
   coronary inflammation markers,
   pre-thrombotic status markers,
   thrombosis markers,
   ischemic markers,
   necrosis markers, and
   myocardial dysfunction markers,
deducing from the measured presence and/or level of said biochemical markers, the probability that the patient will experience a vascular event.

### SUMMARY OF THE INVENTION

The present invention relates to a method for the prognosis of a vascular event in a patient suspected of being at risk for a vascular event, said patient presenting in particular:
no elevation of the ST segment as seen on an electrocardiogram, and/or
a normal level of at least one marker of necrosis, in particular of myocardial necrosis,
   wherein the presence and/or the level of at least two different biochemical markers, in particular three, four, five, six, seven, or eight, different biochemical markers, is measured in a biological sample of said patient, said biochemical markers being selected from the group consisting of the following classes of markers:
atherosclerotic plaque unstability markers,
atherosclerotic plaque disruption markers,
coronary inflammation markers,
pre-thrombotic status markers,
thrombosis markers,
ischemic markers,
necrosis markers, and
myocardial dysfunction markers,
   whereby the probability that the patient will experience a vascular event is deduced from the measured presence and/or level of the biochemical markers.

The present invention also relates to a method for diagnosing acute coronary syndrome in a patient presenting in particular:
no elevation of the ST segment as seen on an electrocardiogram, and/or
a normal level of at least one necrosis marker, in particular a myocardial necrosis marker,
   comprising measuring the presence and/or the level of at least two different biochemical markers, in particular at least three, more particularly at least four, and even more particularly at least five different biochemical markers, in a biological sample of said patient, said biochemical markers being selected from the group consisting of the following classes of markers:
atherosclerotic plaque unstability markers,
atherosclerotic plaque disruption markers,
coronary inflammation markers,
pre-thrombotic status markers,
thrombosis markers,
ischemic markers,
necrosis markers, in particular myocardial necrosis markers, and
myocardial dysfunction markers,
   whereby it is determined if said patient suffers from ACS.

In an embodiment of the above-defined prognosis and diagnosis methods the patient presents:
no elevation of the ST segment as seen on an electrocardiogram, and
a normal level of at least one necrosis marker, in particular a myocardial necrosis marker.

In another embodiment of the above-defined prognosis and diagnosis methods, the patient further presents a chest pain.

In another embodiment of the above defined prognosis and diagnosis methods the presence or the level of the necrosis marker, in particular the myocardial necrosis marker, the level of which is normal in said patient to which the prognosis or diagnosis methods are applied, is used for deducing the probability that the patient will experience a vascular event or for determining if said patient suffers from ACS.

In yet another embodiment of the above-defined prognosis and diagnosis methods, the patient presents a normal cardiac Troponin level, in particular a normal cardiac Troponin I (cTnI) or cardiac Troponin T (cTnT) level, and/or a normal CK level.

In a further embodiment of the above-defined prognosis and diagnosis methods, the patient presents no myocardial necrosis.

The present invention also relates to a method for determining the condition of an individual suspected of having an ACS comprising measuring the presence and/or levels of at least two different biochemical markers in a biological sample of said patient, said biochemical markers being selected from the group consisting of the following combinations of markers:

proBNP and PAPP-A, and optionally a marker selected from the group consisting of CRP, IL-6, MPO, sCD40L, D-Dimer, and BNP;

proBNP, IL-6, and a marker selected from the group consisting of CRP, sCD40L, and BNP;

proBNP and BNP, and optionally a marker selected from the group consisting of CRP, sCD40L, and MPO;

BNP or proBNP, in particular BNP, and CRP, and optionally a marker selected from the group consisting of IL-6, MPO, PAPP-A, and D-Dimer;

proBNP or BNP, in particular proBNP, sCD40L, and optionally a marker selected from the group consisting of CRP and MPO;

CRP and IL-6;

BNP or proBNP, in particular BNP, and IL-6, and optionally CRP and MPO;

proBNP, PAPP-A, CRP, BNP, D-Dimer, IL-6, sCD40L, and MPO;

whereby the condition of the individual is determined.

In an embodiment of the above-defined method for determining the condition of an individual suspected of having an ACS, the condition is selected from the group constituted of:

no ACS, and

unstable angina.

In another embodiment of the above-defined method for determining the condition of an individual suspected of having an ACS, (i) the level of at least one necrosis marker, in particular a myocardial necrosis marker, of the individual and (ii) the presence or absence of an elevation of the ST segment as seen on an electrocardiogram of the patient are determined.

In yet another embodiment of the above-defined method for determining the condition of an individual suspected of having an ACS, the necrosis marker can be cardiac Troponin I or cardiac Troponin T.

The present invention also relates to a kit intended for diagnosing ACS or for the prognosis of a vascular event in a patient, said kit comprising at least three, in particular three, four, five, six, seven, or eight specific ligands respectively to at least three, in particular three, four, five, six, seven, or eight, different biochemical markers, selected from the group consisting of the following classes of markers:

atherosclerotic plaque unstability markers,

atherosclerotic plaque disruption markers,

coronary inflammation markers,

pre-thrombotic status markers,

thrombosis markers,

ischemic markers,

necrosis markers, in particular myocardial necrosis markers,

myocardial dysfunction markers.

In an embodiment, the above defined kit comprises one specific ligand to cardiac Troponin, in particular to cardiac Troponin I or to cardiac Troponin T.

### DETAILED DESCRIPTION OF THE INVENTION

As intended herein “prognosis” relates to methods for predicting the outcome of a pathology or for predicting the probability or likeliness that an adverse event will occur in a patient.

As intended herein “vascular event” relates to a vascular-related, in particular a cardiovascular-related, health-threatening or pathological event in a patient organism. Vascular events according to the invention are in particular selected from the group consisting of vascular-related death, myocardial infarction, congestive-heart failure and vascular-related hospitalization, revascularization procedure.

As intended herein “diagnosis” relates to method for determining the pathology afflicting a patient.

As intended herein acute coronary syndrome (ACS) relates to a variety of clinical symptoms in particular caused by acute myocardial ischemia. It is notably defined in Apple et al. (2005) Clinical Chem 51:810-824.

Preferably, as intended herein, an individual will be said no to present an ACS if is healthy and/or if he presents a normal level of a necrosis marker, in particular a myocardial necrosis factor, no elevation of the ST segment, and a non-pathological electrocardiogram.

As intended herein “no elevation of the ST segment as seen on an electrocardiogram” or “no elevation of the ST segment” means that the patient's electrocardiogram either presents a depression of the ST segment or a normal ST segment.

As intended herein a “normal level” of a marker means that the concentration of the marker in the biological sample is within the norm cut-off values for that marker. The norm is dependant on the biological sample type and on the method used for measuring the concentration of the marker in the biological sample. Norms for a given marker are well known to the man skilled in the art. In particular, a normal level for a given marker means that the marker is substantially absent, that is the level measured for that marker is below a cut-off value defining the minimal level for that marker to be considered significantly present.

As intended herein a “necrosis marker”, in particular a “myocardial necrosis marker”, relates to a macromolecule which is released from myocardial tissues upon myocardial necrosis, notably after myocardial ischemia, for example. Necrosis marker notably encompasses cardiac Troponin, in particular cardiac Troponin I and T (cTnI and cTnT), myoglobin, as well as creatine kinase (CK). As regards CK it is preferred that the MB isoform is measured.

Preferably, to be regarded as normal, measured levels for cardiac Troponin I should not exceed the 99^{th} percentile of values for a reference group during at least 24 hours.

Preferably also, to be regarded as normal, measured levels for the MB isoform of CK should not exceed the 99^{th} percentile of values for a gender-specific reference control group on two successive samples.

As intended herein the levels of the markers are measured using any method liable to yield the concentration of the marker in the biological sample. Such a method notably encompasses mass spectroscopy (in particular for measuring CK-MB levels), but also ligand-based methods such as immunological method.

In particular the levels of the biochemical markers are measured using ligands specific for said biochemical markers.

A “ligand” relates to a molecule which has binding affinity towards a marker. In particular it is preferred that the ligand binds to the marker in a specific manner, that is the ligand preferentially binds to the marker it is specific for as compared to other components of the biological sample.

The specific ligands are in particular selected from the group consisting of:
polyclonal, monoclonal and recombinant antibodies, or fragments thereof, such as Fab fragments, F(ab′)₂ fragments and scFv fragments,
phage antibodies (PhAbs),
llama and camel antibodies,
aptamers.

Preferably, the specific ligands are monoclonal antibodies.

As intended herein the expression “biological sample” encompasses all samples which can be taken from a patient, such as tissue biopsies or samples of body fluids. However, it is preferred in the Invention that the samples are blood-based samples, such as whole-blood samples, serum or plasma samples. In case of blood based samples any anticoagulant, such as EDTA, citrate or heparin, can be added.

Furthermore, the levels of the markers can be measured from different samples taken from a same patient or from a unique sample.

Moreover, the presence or the levels of markers in the samples can be determined individually, in simplex assays, or optionally, the levels of some or all of the markers can be determined simultaneously, in multiplex assays.

The presence or the level of a marker can be determined once or repeatedly, notably according to a predefined time-course.

It is preferred that the presence or the level of a marker is determined using an automated assay system.

As intended herein:
atherosclerotic plaque unstability markers relate to markers of which the presence or the level is altered in case of unstable atherosclerotic plaque. In particular such markers are present or their level increases in case of unstable atherosclerotic plaque;
atherosclerotic plaque disruption markers relate to markers of which the presence or the level is altered in case of atherosclerotic plaque disruption. In particular such markers are present or their level increases in case of atherosclerotic plaque disruption;
coronary inflammation markers relate to markers of which the presence or the level is altered in case of coronary inflammation. In particular such markers are present or their level increases in case of coronary inflammation;
pre-thrombotic status markers relate to markers of which the presence or the level is altered in case of pre-thrombosis. In particular such markers are present or their level increases in case of pre-thrombosis;
thrombosis markers relate to markers of which the presence or the level is altered in case of thrombosis. In particular such markers are present or their level increases in case of thrombosis;
ischemic markers relate to markers of which the presence or the level is altered in case of myocardial ischemia. In particular such markers are present or their level increases in case of myocardial ischemia;
necrosis markers, in particular myocardial necrosis markers, relate to markers of which the presence or the level is altered in case of myocardial necrosis. In particular such markers are present or their level increases in case of myocardial necrosis;
myocardial dysfunction markers relate to markers of which the presence or the level is altered in case of myocardial dysfunction. In particular such markers are present or their level increases in case of, myocardial dysfunction.

Such markers are notably described in Apple et al. (2005) Clin. Chem. 51:810-824.

As intended herein two or more of the markers can belong to a same class of markers. However, in a preferred embodiment of the above-defined prognosis and diagnosis methods, the biochemical markers do not belong to the same class of markers.

In another preferred embodiment of the above-defined prognosis and diagnosis methods, the following combinations of markers are used:

| | Markers | | | | | | | |
|---|---|---|---|---|---|---|---|---|
| | | | | pre- | | | | |
| | plaque | plaque | coronary | thrombotic | | | | myocardial |
| Combination | unstability | disruption | inflammation | status | thrombosis | ischemic | necrosis | dysfunction |
| 1 | x | x | x | | | | | |
| 2 | x | x | | x | | | | |
| 3 | x | x | | | x | | | |
| 4 | x | x | | | | x | | |
| 5 | x | x | | | | | x | |
| 6 | x | x | | | | | | x |
| 7 | x | | x | x | | | | |
| 8 | x | | x | | x | | | |
| 9 | x | | x | | | x | | |
| 10 | x | | x | | | | x | |
| 11 | x | | x | | | | | x |
| 12 | x | | | x | x | | | |
| 13 | x | | | x | | x | | |
| 14 | x | | | x | | | x | |
| 15 | x | | | x | | | | x |
| 16 | x | | | | x | x | | |
| 17 | x | | | | x | | x | |
| 18 | x | | | | x | | | x |
| 19 | x | | | | | x | x | |
| 20 | x | | | | | x | | x |
| 21 | x | | | | | | x | x |
| 22 | | x | x | x | | | | |
| 23 | | x | x | | x | | | |
| 24 | | x | x | | | x | | |
| 25 | | x | x | | | | x | |
| 26 | | x | x | | | | | x |
| 27 | | x | | x | x | | | |
| 28 | | x | | x | | x | | |
| 29 | | x | | x | | | x | |
| 30 | | x | | x | | | | x |
| 31 | | x | | | x | x | | |
| 32 | | x | | | x | | x | |
| 33 | | x | | | x | | | x |
| 34 | | x | | | | x | x | |
| 35 | | x | | | | x | | x |
| 36 | | x | | | | | x | x |
| 37 | | | x | x | x | | | |
| 38 | | | x | x | | x | | |
| 39 | | | x | x | | | x | |
| 40 | | | x | x | | | | x |
| 41 | | | x | | x | x | | |
| 42 | | | x | | x | | x | |
| 43 | | | x | | x | | | x |
| 44 | | | x | | | x | x | |
| 45 | | | x | | | x | | x |
| 46 | | | x | | | | x | x |
| 47 | | | | x | x | x | | |
| 48 | | | | x | x | | x | |
| 49 | | | | x | x | | | x |
| 50 | | | | x | | x | x | |
| 51 | | | | x | | x | | x |
| 52 | | | | x | | | x | x |
| 53 | | | | | x | x | x | |
| 54 | | | | | x | x | | x |
| 55 | | | | | x | | x | x |
| 56 | | | | | | x | x | x |

According to a particularly preferred embodiment of the above-defined prognosis and diagnosis methods:
the atherosclerotic plaque unstability marker is selected from the group consisting of MPO, sCD40L, IL-6, PAPP-A and Choline, in particular whole blood Choline;
the atherosclerotic plaque disruption marker is selected from the group consisting of MPO, sCD40L, MMPs, Choline, in particular whole blood Choline, and PAPP-A;
the coronary inflammation marker is selected from the group consisting of MPO, sCD40L, CRP and IL-6;
the pre-thrombotic status marker is selected from the group consisting of sCD40L and fibrinogen;
the thrombosis marker is selected from the group consisting of D-Dimers;
the ischemic marker is selected from the group consisting of IMA, cTnI, cTnT, and FABP;
the necrosis marker, in particular the myocardial necrosis marker, is selected from the group consisting of cTnI, cTnT, CK, Myoglobin and FABP;
the myocardial dysfunction mariner is selected from the group consisting of BNP, NT-proBNP and proBNP.

MPO (myeloperoxydase) is notably described in Baldus et al., Circ. 2003; 108:1440-1445, Brennan et al., N Engl J Med 2003; 349:1595-604, Brennan et al., Current opinion in lipidology 2003; 14:353-359, Nambi et al., Curr Ather Reports 2005, 7:127-131, Nicholls et al., Arterioscler Thromb Vasc Biol. 2005; 25:1-9 and Zhang et al. JAMA 2001; 286:2136-2142.

MMPs (matrix metalloproteinases), in particular MMP-9, are notably described in Jones et al., Cardiovasc Res 2003; 59:812-23.

Choline is notably described in Apple et al., Clin. Chem. 2005; 51:810-824.

sCD40L (soluble form of CD40 ligand) is notably described in Aukrust et al., Circ. 1999; 100:614-620; Heeschen et al., N Engl J Med 2003; 348:1104-11; Peng et al., Clin Chim Acta. 2002; 319:19-26; Tayebjee et al., Am J Cardiol 2005; 96:339-345; Varo et al., Circ. 2003; 108:1049-1052.

IL-6 (interleukin 6) is notably described in Aggarwal et al. Journal of Thrombosis and Thrombolysis 2003; 15: 25-31, 2003; Biasucci et al. Circ. 1999; 99:855-860; Ikeda et al. Clin. Cardiol. 2001; 24:701-704; Lubrano et. al., J of Clin Lab Anal 2005; 19:110-114; Ridker et al., N Engl J. Med. 2000; 342:836-43.

PAPP-A (pregnancy associated plasma protein A) is notably described in Bayes-Genis et al., N Engl J Med 2001; 345:1022-9; Heeschen et al., J Am Coll Cardiol 2005; 45:229-37; Khosravi et al., Clinical Biochemistry 2002; 35:531-538; Laterza et al., Clin Chim Acta 2004; 348:163-169; Liu et al., Chin med j. 2005; 118:1827-1829. Lund et al., Circulation 2003; 108:1924-1926. Qin et al., Scand Cardiovasc J 36; 358-361, 2002; Qin et al., Clin Chem. 2005; 51:75-83; Qin et al., Clin Chem 2006; 52:398-404.

D-Dimer is notably described in Bayes-Genis et al., Am Heart J 2000; 140:379-84. Danesh et al., Circ. 2001; 103:2323-2327; Heim et al., Clinical Chemistry 2004; 50:1136-1147; Keeling et al. British Journal of Haematology, 2004; 124:15-25; Shitrit et al., American Journal of Hematology 2004; 77:147-150; Shitrit et al. American Journal of Hematology 2004; 76:121-125.

CRP (C reactive protein) is notably described in Bermudez et al. Arterioscler Thromb Vasc Biol. 2002; 22:1668-1673; Body et al., International Journal of Cardiology 2005; 98:277-283; Ferreiro et al., Circ. 1999; 100:1958-1963; Haverkate et al., Lancet 1997; 349: 462-66; Lowe et al., Journal of Thrombosis and Hemostasis 2003; 1:2312-2316; Luc et al., Arterioscler Thromb Vasc Biol. 2003; 23:1255-1261; Maier et al. Circ, 2005; 111:1355-1361; Pepys et al., J. Clin. Invest. 2003 111:1805-1812; Ridker et al., Am Heart J 2004; 148:S19-26; Roberts et al. Clin Chem 2001; 47:418-425; Tanaka et al., J Am Coll. Cardiol 2005; 45:1594-9; Tzoulaki et al. Circ. 2005; 112:976-983.

Fibrinogen is notably described in Saigo et al., Progress in Cardiovascular Diseases 2004; 46:524-538.

IMA (ischemia modified albumin) is notably described in Jaffe Cardiol Clin 2005 23:453-465.

FABP (fatty acid binding protein) is notably described in Suzuki et al. Int. Heart J 2005 46:601-606.

Myoglobin is notably described in Jaffe Cardiol Clin 2005 23:453-465.

cTnI is notably described in Babuin et al., CMAJ 2005; 173:1191-1202; Bodi et al., International journal of Cardiology 2005; 98:277-283; Collinson et al., Ann clin biochem 2001; 38:423-449; Heeschen et al., Clin Biochem 2000; 33:359-368; Kamineni et al., Progress in cardiovascular diseases 2004; 46:379-392; Maier et al., Clin chim acta 1996; 45:19-38; McDonough et al., Progress in cardiovascular diseases 2004; 47:207-216; Mockel et al., Clin chim acta 2000; 293:139-155; Newby et al., Progress in cardiovascular diseases 2004; 46:404-416; Panteghini et al., Clin Chem Lab Med. 2001; 39:175-179. Rao et al. Am J cardiol 2003; 91:936-940; Ritter et al. Clin Chem. 2004; 50:112-119; Rosalki et al. Clin Chem 2004; 60:2205-2213; Scirica et al. Progress in cardiovascular diseases 2004; 47:177-188.

cTnT is notably described in Collinson et al., Med Princ Pract 2007; 16:247-61; Anwaruddin et al., J Am Coll Cardiol 2007; 49:279-89; Eisenman, Expert Rev Cardiovasc Ther 2006; 4:509-14; Engel et al., Mol Diagn Ther 2007; 11:109-16; Cameron et al., Clin Chem Acta 2007; 376:168-73; Goodacre et al. BMC Emerg Med 2005, 5:6; Heeschen et al., Circulation 2004; 110:3206-12; Kaul et al., J Am Coil Cardiol 2003; 41:371-80.

BNP (B natriuretic peptide), proBNP and NT-proBNP are notably described in Alehagen et al. International Journal of Cardiology 2005; 100:125-133; Bassan et al., European Heart Journal 2005; 26:234-240; De lemos et al., N Engl J Med 2001; 345:1014-21; de Lemos et al., Rev Cardiovasc Med 2003; 4:S37-S46; Fonarow et al., Rev Cardiovasc Med 2003; 4:S20-S28; Galvani et al., The European Journal of Heart Failure 2004; 6:327-333; Gibler et al., Rev Cardiovasc Med 2003; 4:S47-S55; Jernberg et al., European Heart Journal 2004; 25:1486-1493; Mega et al., J Am Coll Cardiol 2004; 44:335-9; Na Hong et al., Circ J 2005; 69:1472-1476; Sabatine et al., J Am Coll Cardiol 2004; 44:1988-95; Sokoll et al. Clin Chem Lab Med 2004; 42:965-972; Wiviott et al., Clin Chim Acta 2004; 346:119-128; Giuliani et al., Clinical Chemistry 2006; 52:1054-1061; Seferian et al, Clinical Chemistry 2007; 53, 86673; Lam et al, JACC 2007; 49; 1193-202; Liang et al, JACC 2007; 49; 1071-8.

In a particularly preferred embodiment of the above defined prognosis and diagnosis methods, the following combinations of biochemical markers are used:

| Com- | Markers | | | | | | | |
|---|---|---|---|---|---|---|---|---|
| bina- | | | IL- | PAPP- | | D- | | |
| tions | MPO | sCD40L | 6 | A | CRP | Dimer | cTnl | proBNP |
| 1 | x | x | x | | | | | |
| 2 | x | x | | x | | | | |
| 3 | x | x | | | x | | | |
| 4 | x | x | | | | x | | |
| 5 | x | x | | | | | x | |
| 6 | x | x | | | | | | x |
| 7 | x | | x | x | | | | |
| 8 | x | | x | | x | | | |
| 9 | x | | x | | | x | | |
| 10 | x | | x | | | | x | |
| 11 | x | | x | | | | | x |
| 12 | x | | | x | x | | | |
| 13 | x | | | x | | x | | |
| 14 | x | | | x | | | x | |
| 15 | x | | | x | | | | x |
| 16 | x | | | | x | x | | |
| 17 | x | | | | x | | x | |
| 18 | x | | | | x | | | x |
| 19 | x | | | | | x | x | |
| 20 | x | | | | | x | | x |
| 21 | | x | x | x | | | | |
| 22 | | x | x | | x | | | |
| 23 | | x | x | | | x | | |
| 24 | | x | x | | | | x | |
| 25 | | x | x | | | | | x |
| 26 | | x | | x | x | | | |
| 27 | | x | | x | | x | | |
| 28 | | x | | x | | | x | |
| 29 | | x | | x | | | | x |
| 30 | | x | | | x | x | | |
| 31 | | x | | | x | | x | |
| 32 | | x | | | x | | | x |
| 33 | | x | | | | x | x | |
| 34 | | x | | | | x | | x |
| 35 | | | x | x | x | | | |
| 36 | | | x | x | | x | | |
| 37 | | | x | x | | | x | |
| 38 | | | x | x | | | | x |
| 39 | | | x | | x | x | | |
| 40 | | | x | | x | | x | |
| 41 | | | x | | x | | | x |
| 42 | | | x | | | x | x | |
| 43 | | | x | | | x | | x |
| 44 | | | | x | x | x | | |
| 45 | | | | x | x | | x | |
| 46 | | | | x | x | | | x |
| 47 | | | | x | | x | x | |
| 48 | | | | x | | x | | x |
| 49 | | | | | x | x | x | |
| 50 | | | | | x | x | | x |
| 51 | | | | | | x | x | x |

In another preferred embodiment of the above defined prognosis and diagnosis methods, the following combinations of biochemical markers are used:

proBNP and PAPP-A, and optionally a marker selected from the group consisting of CRP, IL-6, MPO, sCD40L, D-Dimer, and BNP;

proBNP, IL-6, and a marker selected from the group consisting of CRP, sCD40L, and BNP;

proBNP and BNP, and optionally a marker selected from the group consisting of CRP, sCD40L, and MPO;

BNP or proBNP, in particular BNP, and CRP, and optionally a marker selected from the group consisting of IL-6, MPO, PAPP-A, and D-Dimer;

proBNP or BNP, in particular proBNP, sCD40L, and optionally a marker selected from the group consisting of CRP and MPO;

CRP and IL-6;

BNP or proBNP, in particular BNP, and IL-6, and optionally CRP and MPO;

proBNP, PAPP-A, CRP, BNP, D-Dimer, IL-6, sCD40L, and MPO.

whereby the condition of the individual is determined.

Particularly, preferred are the following combinations:

| proBNP | BNP | CRP | IL-6 | MPO | sCD40L | PAPP-A |
|---|---|---|---|---|---|---|
| | x | x | | | | |
| | x | | x | | | |
| | | x | x | | | |
| x | | | | | | x |
| x | x | | | | | |
| x | | | | | x | |

| | | | | | | PAPP- | | D- |
|---|---|---|---|---|---|---|---|---|
| proBNP | BNP | CRP | IL-6 | MPO | sCD40L | A | cTnl | Dimer |
| | x | x | x | | | | | |
| | | x | x | | | | x | |
| x | x | x | | | | | | |
| x | x | | | | | x | | |
| | x | x | | x | | | | |
| x | x | | | | x | | | |
| x | x | | x | | | | | |
| x | x | | | x | | | | |
| x | | x | | | x | | | |
| x | | | x | | x | | | |
| x | | | | x | x | | | |
| x | | x | x | | | | | |
| | x | x | | | | x | | |
| x | | | | | | x | | x |
| | x | x | | | | | | x |
| x | | | | x | | x | | |
| x | | x | | | | x | | |
| x | | | | | x | x | | |
| x | | | x | | | x | | |

BNP, IL-6, CRP, and MPO;
proBNP, PAPP-A, CRP, BNP, D-Dimer, IL-6, sCD40L, and MPO.

Besides, in the above-defined prognostic method, it is more particularly preferred that the following combinations be used:

| | |
|---|---|
| a₁ | BNP, CRP and proBNP |
| b₁ | BNP and proBNP |
| c₁ | BNP and CRP |
| d₁ | BNP, proBNP and PAPP-A |
| e₁ | BNP, CRP and MPO |
| f₁ | BNP, proBNP and sCD40L |
| g₁ | BNP, IL-6, CRP and MPO |
| h₁ | BNP, proBNP and IL-6 |
| i₁ | BNP, proBNP and MPO |
| j₁ | proBNP, sCD40L and CRP |
| k₁ | proBNP, sCD40L and IL-6 |
| l₁ | proBNP, sCD40L and MPO |
| m₁ | proBNP and sCD40L |

Indeed, these combinations when applied to an asymptomatic population (i.e. a population with a normal cTnI level and a normal ECG) enable to distinguish healthy individuals from individuals which have presented a chest pain suggestive of a vascular event. Individuals which have presented a chest pain suggestive of a vascular event are believed to be more prone to presenting a future vascular event.

Accordingly, the above combinations are of high prognostic value in testing asymptomatic populations for the probability of a future vascular event.

In particular, when combination a₁ is used, an asymptomatic individual will be said to present an increased probability of having a future vascular event, with respect to a healthy individual if:

a₁) 0.109 BNP−0.019 CRP−0.022 proBNP≧0.116,

Likewise, the following equations apply to combinations b₁ to m₁:

b₁) 0.108 BNP−0.022 proBNP≧0.126

c₁) 0.031 BNP+0.079 CRP≧0.37

d₁) 0.109 BNP−0.022 proBNP−0.003 PAPP-A≧0.007

e₁) 0.031 BNP+0.102 CRP−0.004 MPO≧0.016

f₁) 0.107 BNP−0.022 proBNP+0.074 sCD40L≧0.215

g₁) 0.031 BNP+0.041 IL-6+0.078 CRP−0.004 MPO≧0.112

h₁) 0.115 BNP−0.092 IL-6−0.023 proBNP≧0.118

i₁) −0.002 MPO+0.110 BNP−0.023 proBNP≧0.1

j₁) 0.156 sCD40L+0.112 CRP−0.001 proBNP≧0.151

k₁) 0.12 sCD40L+0.054 IL-6+0.001 proBNP≧0.23

l₁) 0.107 sCD40L+0.001 MPO+0.001 proBNP≧0.216

m₁) 0.105 sCD40L+0.001 proBNP≧0.107

where the following units are applied to the concentrations of each marker: BNP, pg/ml; proBNP, pg/ml; MPO, ng/ml; D-Dimer, ng/ml; CRP, mg/l; IL-6, pg/ml; PAPP-A, ng/ml; sCD40L, ng/ml.

It is also preferred, in the above-defined diagnostic method, that the following combinations be used to differentiate individuals having an ACS, in particular having unstable angina, from individuals who do not have an ACS, in a population having a normal cTnI level and no elevation of the ST-segment:

| | |
|---|---|
| a₂ | BNP and CRP |
| b₂ | proBNP, CRP and IL-6 |
| c₂ | BNP, CRP and IL-6 |
| d₂ | BNP, CRP and PAPP-A |
| e₂ | proBNP, CRP and PAPP-A |
| f₂ | proBNP, MPO and PAPP-A |
| g₂ | proBNP, BNP and CRP |
| h₂ | proBNP, D-Dimer and PAPP-A |
| i₂ | proBNP, IL-6 and PAPP-A |
| j₂ | proBNP, sCD40L and PAPP-A |
| k₂ | proBNP and PAPP-A |
| a₃ | BNP, D-Dimer and CRP |
| b₃ | BNP, IL-6 and CRP |
| c₃ | PAPP-A, MPO and proBNP |
| d₃ | PAPP-A, CRP and proBNP |
| e₃ | PAPP-A, sCD40L and proBNP |
| f₃ | PAPP-A, IL-6 and proBNP |
| g₃ | PAPP-A, BNP and proBNP |
| h₃ | PAPP-A and proBNP |
| i₃ | IL-6, CRP and proBNP |
| j₃ | BNP, proBNP, D-Dimer, IL-6, |
| | CRP, PAPP-A, sCD40L, MPO |

and/or

More particularly, combinations a₂ to k₂ are useful to differentiate unstable angina individuals from healthy individuals, and combinations a₃ to j₃ are useful to differentiate unstable angina individuals having a normal cTnI level and no elevation of the ST segment but having experienced a chest pain suggestive of a vascular event.

Thus, an individual having a normal cTnI level and no elevation of the ST segment will be considered as suffering from unstable angina if the following equations apply:

0.002BNP+0.078CRP≧0.219 a₂)

0.001proBNP+0.133CRP−0.07IL-6≧0.922 b₂)

0.005BNP+0.137CRP−0.208IL-6≧0.132 c₂)

0.003BNP+0.077CRP−0.002PAPP-A≧0.162 d₂)

−0.01PAPP-A+0.146CRP+0.001proBNP≧0.079 e₂)

−0.01PAPP-A+0.003MPO+0.001proBNP≧0.308 f₂)

−0.002BNP+0.130CRP+0.001proBNP≧0.504 g₂)

−0.007PAPP-A+0.001D-Dimer+0.001proBNP≧0.115 h₂)

−0.008PAPP-A+0.111IL-6+0.001proBNP≧0.111 i₂)

−0.006PAPP-A+0.021sCD40L+0.001proBNP≧0.031 j₂)

−0.006PAPP-A+0.001proBNP≧0.02 k₂)

0.003BNP−0.001D-Dimer−0.007CRP≧0 a₃)

0.004BNP+0.041CRP−0.127IL-6≧0 b₃)

−0.027PAPP-A+0.005MPO+0.002proBNP≧0.296 c₃)

−0.03PAPP-A+0.155CRP+0.002proBNP≧−0.252 d₃)

−0.016PAPP-A−0.156sCD40L+0.001proBNP≧−0.061 e₃)

−0.016PAPP-A−0.034IL-6+0.001proBNP≧−0.163 f₃)

−0.016PAPP-A−0.001BNP+0.001proBNP≧−0.313 g₃)

−0.015PAPP-A+0.001proBNP≧−0.31 h₃)

−0.06IL-6+0.057CRP+0.001proBNP≧0.342 i₃)

0.016MPO−0.068PAPP-A−0.002D-Dimer+0.011BNP−0.721 j₃)

sCD40L−0.131IL-6+0.249CRP+0.002proBNP≧0.38

where the following units are applied to the concentrations of each marker: BNP, pg/ml; proBNP, pg/ml; MPO, ng/ml; D-Dimer, ng/ml; CRP, mg/l; IL-6, pg/ml; PAPP-A, ng/ml; sCD40L, ng/ml

Conversely, if the equations do not apply, the individual will be said not to suffer from an ACS.

As intended herein, it is preferred for the above-defined prognostic, diagnostic, and determination methods that deduction of the probability that the patient will experience a vascular event, determining if the patient suffers from ACS, or determining the condition of the patient, from the measured presence or level of the markers, is performed using a computer-based system. Even more preferably, the computer-based system is included in the automated assay system.

Depending on the deduced probability that the patient will experience a vascular event, it is within the frame of the present invention that several therapeutically decisions, such as hospitalization or pharmaceutical treatment, are offered to the practitioner using the method according to the invention. In particular if the deduced probability that the patient will experience a vascular event is considered negligible then it is proposed that the patient be sent home; on the contrary, if the deduced probability that the patient will experience a vascular event is considered high then it is proposed that the patient be admitted to hospital.

Probability that the patient will experience a vascular event can be assessed using scoring systems well known to the man skilled in the art.

Such scoring systems are notably described in Sabatine et al. (2002) Circ. 105:1760-1763. For instance, 0 or 1 is affected for each marker in the combination of markers used in the method according to the invention, depending on whether this marker is respectively absent or present or whether the level of this marker is respectively below or above a predetermined cut-off value for normality, and a global sum is calculated for the combination of markers.

Alternatively, values between 0 and 1 can be used using several cut-off values such as described in WO 2004/059293. Besides, weighing factors can be affected to certain particular combinations of levels of markers which reflect a particular physiological setting.

The sum is then compared to predetermined values which are correlated to a probability that the patient will experience a vascular event. These predetermined values can be obtained according to statistical methods well known to the man skilled in the art, such as those described in US 2006/0105419 (see Example 3) or in EP 1 666 881 (see [0126]-[0143]). In particular, these predetermined values can be obtained by using clinical studies wherein the occurrence of a vascular event in a patient within a determined follow-up period, or clinical end-point, is correlated to the level of one or more biochemical markers in said patient at the beginning of the follow-up period.

The biochemical markers which are used in the methods according to the invention are selected according to their statistical significance with respect to various differential analysis methods, such as T-Student test, ANOVA, or Kruskal-Wallis test. The markers can be further selected according to their discriminative capacity through a Receiver Operating Characteristic (ROC) curve analysis well-known to the man skilled in the art (Griner et al. Annals of Internal Medicine 1981; 94:555-600). Finally, the best markers are combined to increase their sensitivity (Se) and their specificity (Sp), for example, by following the general mROC method described by Kramar et al. Revue d'Epidémiologie et Santé Publique 1999; 47:376-383 or with unsupervised learning algorithms such as decision trees, Support Vector Machines (SVMs) or neural networks.

Preferably, the above-defined kit comprises ligands specific respectively to the markers of the combinations of markers selected from the group constituted of:

proBNP and PAPP-A, and optionally a marker selected from the group consisting of CRP, IL-6, MPO, sCD40L, D-Dimer, and BNP;

proBNP, IL-6, and a marker selected from the group consisting of CRP, sCD40L, and BNP;

proBNP and BNP, and optionally a marker selected from the group consisting of CRP, sCD40L, and MPO;

BNP or proBNP, in particular BNP, and CRP, and optionally a marker selected from the group consisting of IL-6, MPO, PAPP-A, and D-Dimer;

proBNP or BNP, in particular proBNP, sCD40L, and optionally a marker selected from the group consisting of CRP and MPO;

CRP and IL-6;

BNP or proBNP, in particular BNP, and IL-6, and optionally CRP and MPO;

proBNP, PAPP-A, CRP, BNP, D-Dimer, IL-6, sCD40L, and MPO.

### Example 1

### Prospective Clinical Study

### Objective

The objective of the clinical study is to make a statistical evaluation of the prognostic value of various combinations of markers by determining the sensibility, the specificity, the positive predictive value and the negative predictive value of each combination. The validation of the results is made by following the occurrence of vascular events in patients (pain recurrence, worsening of the patient's condition to unstable angina pectoris, non-fatal myocardial infarct, fatal myocardial infarct) 3 to 6 months following a first hospital admission for a chest pain.

The automated simultaneous determination in a single patient sample of these combinations of markers is particularly useful for the physician to help him establish risk stratifications for patients with chest pains and with no electrocardiogram (ECG) modification nor cardiac marker elevation.

### Patients

At least 300 patients consulting for chest pains and without elevation of the ST segment nor of cardiac Troponin I level on admission are enrolled in this study.

The following inclusion criteria are used:
the patients are 18 years old and over and present with chest pains without elevation of the ST segment on the ECG performed on admission;
the patients for whom cTnI assays performed on admission are negative.

Moreover, in order to validate the results a wide range of patients either with pathologies characteristic of Acute Coronary Syndrome or not are also selected:

50 myocardial infarcts: with typical prolonged pain and ECG modification and/or an elevation of the biochemical markers (e.g. cTnI).

100 healthy subjects, with no particular cardiovascular risk; this population is heterogeneous as regards age and sex.

Patients presenting risk factors are also included:
Smokers,

Patients with high arterial tension,

Diabetics,

Elderly patients,

Patients with high triglyceride level.

However, patients admitted with the preceding characteristics but with pain for 12 hours or more are not included in the study. Patients with a non-normal ECG which cannot be used to visualize ischemic changes in the ST segment (e.g. patients with pace maker) significant of a myocardial infarct or with an elevated level of cTnI on admission are not included in the study either.

Moreover, subjects with inflammatory diseases (CRP>15 mg/l) and tumors, but also subjects having undergone surgery or a major trauma in the preceding month are not included in the study. Furthermore, patients having known thrombosis disorders or cardiovascular pathologies, or a myocardial infarct in the 3 weeks preceding admission are also excluded from the present study.

Patients with hepatic or renal disorders are not included in the study due to the risk of obtaining significantly elevated levels for the markers having a rapid renal clearance in healthy patients.

Pregnant women are also excluded from the study.

The following information on the included patients are collected: age, sex, smoking habits, diabetic status, pain characteristics (pain localization and strength, duration, recurrence), ECG characteristics on admission, admission time, cTnI assay results on admission, final diagnostic. Hormonal substitution treatment for menopaused women is also recorded.

### Sampling

For each patient, 2 ml of venous blood are collected in standard EDTA and dry tubes. Each tube is labelled with a code identifying the patient, date and time of sampling, and time lapsed from admission to sampling.

### Assays

Assays for cTnI, IL-6, hsCRP, MPO, PAPP-A, D-Dimer, sCD40L, BNP and proBNP are conducted on the samples with methods known in the art.

The levels of the various markers for each patient will then be correlated to the putative occurrence of one or several vascular events in said patient, such as pain recurrence, worsening of the patient's condition to unstable angina pectoris, non-fatal myocardial infarct or fatal myocardial infarct, 3 to 6 months after sampling, according to statistical methods well known to the man skilled in the art, such as an analysis-of-variance (ANOVA). As such, it can be determined whether a significant relation exists between the studied variables (i.e. the studied clinical markers) and the vascular events.

### Example 2

### Clinical Study

### Materials and Methods:

Patient population: The study population consisted of 32 patients suspected for acute coronary syndrome (ACS) and discharged from the emergency department (rule out population with no ACS), 22 patients diagnosed for unstable angina, 38 patients with acute myocardial infarction and 47 normal healthy volunteers. All included healthy volunteer and patient participants provided written informed consent.

Patients admitted in the emergency medicine department with symptoms such as acute chest pain with suspected acute coronary syndrome were included through a study protocol approved by the institutional review board of Brabois Hospital (Central University Hospital CHU, Nancy, France). Following admission, an electrocardiogram (12-lead ECG) and blood level of cardiac Troponin I assay (cTnI, Stratus CS troponin testing, Dade Behring, USA), were performed to discriminate proven unstable patients from patients with no ACS (rule out for acute coronary syndrome). Patients without elevation of ST-segment and normal plasma troponin levels (<0.04 ng/ml) were included and classified as unstable patients. Patients presenting chest pain and ischemia type discomfort but not diagnosed for ACS (i.e. presenting a normal electrocardiogram) were defined as rule out population and discharged from the emergency department. One group composed of patients diagnosed for myocardial infarction with ST-elevation segment and/or elevated plasma cardiac troponin I (>0.04 ng/ml) was also included in the study (38 left over EDTA plasma samples available from Hospital of Poissy—Saint Germain en Laye, France). For comparison, control EDTA plasmas from healthy volunteers (25 males and 22 females) were provided by Teragenix Corporation (US). EDTA blood samples from all groups were centrifuged and stored at −80° C. for multimarker panel determination.

For rule out population, unstable and myocardial infarction groups, demographic characteristics and a series of cardiovascular risk factors were recorded, as smoker status, diabetes mellitus, hypertension, total cholesterol level and renal dysfunction. The demographic characteristics of all patients are summarized in Table 1.

### Analytical Methods: Biomarker Measurement

Eight biomarkers were selected for their implication in myocardial damage and dysfunction, inflammation and plaque rupture and were evaluated in plasmas from controls and patients admitted for suggestive ACS comprising myocardial infarction. A series of 8 biochemical variables were measured in all EDTA plasma as followed. Cardiac Troponin I (cTnI, Access AccuTnI, Beckman Coulter) to validate preliminary Troponin I testing and inclusion criteria at emergency admission, B-type natriuretic (BNP) peptide (Biosite Incorporated, San Diego, Calif.), proBNP (Bio-Rad RUO ELISA assay, Clin Chem 2006; 52(6):1054-1061), Interleukin-6 (IL-6, RD systems), D-Dimer (Hyphen Biomed, France) and CRP by nephelometry (BNII, Dade Behring inc., USA) were assessed. Pregnancy associated plasma protein A (PAPP-A) and myeloperoxidase (MPO) were measured in human plasmas by a two-site sandwich immunoassay using monoclonal mouse anti-human PAPP-A and MPO antibodies respectively (HyTest Ltd, Finland). All EDTA plasma having cTnI levels higher than 0.04 ng/ml were excluded from the analysis in rule out no ACS and unstable patients. All sample analysis were performed without clinical information by blinded operator. Baseline levels for each biomarker are presented in Table 2 for all patient populations.

### Statistical Analysis

The marker diagnostic performance could be characterised by: sensitivity, which represents its ability to detect the diseased population and specificity which represents its ability to detect the control population. The results of the evaluation of a diagnostic test can be summarised in a 2×2 contingency table comparing these two well-defined populations. By forming a cut-off, the two populations could be classified into categories according to the results of the test, categorised as either positive or negative. So for a particular marker, a subjects with a positive test result among the “cases” population (the “True Positive”: TP) and b subjects with a positive test result among the “controls” population (the “True Negative”. TN) are observed. In the same fashion, c subjects with a negative test result among the cases (the “False Positive”: FP) and d subjects with a negative test result among the controls (the “False Negative”: FN) are observed.

Sensitivity is defined as TP/(TP+FN); which we refer to as the “true positive rate”. It is calculated in the sample of cases. The sensitivity (Se) of a test at a particular cut-off thus corresponds to the proportion of positive results among the cases.

Specificity is defined as TN/(TN+FP); which we refer to as the “true negative rate”. It is calculated in the sample of controls. The specificity (Sp) of a test at a particular cut-off thus corresponds to the proportion of negative results among the controls.

The sensibility and specificity are calculated for a defined cutoff. The marker concentrations with the highest diagnostic accuracy (minimal false-negative and false positive results) are selected as a defined cutoff or threshold.

AUC (Area Under the Curve) illustrates the overall index for the diagnostic performance of the respective single markers or a combination of markers.

Receiving operating characteristic (ROC) analysis was performed for single biomarker and for multimarker combination (multiple ROC, mROC), when comparing rule out no ACS patients to unstable patients or myocardial infraction patients with mROC software well described by Kramar et al. (Comput. Methods Programs Biomed. 2001; 66:199-207; Revue d'Epidémiologie et SantéPublique 1999; 47:376-83). MROC is a dedicated program calculating the linear combination (Su et al. Journal of the American Statistical Association 1993; 88:1350-1355), to maximize the AUC (Area Under the Curve) ROC for all markers selected and also for all two and three marker combinations among the selected markers (Staack et al. BMC Urology 2006; 6:1-12). The equation for the respective combination is provided and can be used as a new virtual marker. For a marker combination and for a sample selected, the cut-off is the result value of the linear equation corresponding and calculated by the mROC program:

Marker combination cut-off a×Marker₁+b×Marker₂+c×Marker₃, where a, b, c are calculated coefficients and Marker_{1,2,3} are individual marker cut-offs.
Data are as means±standard deviation (SD). All data distributions are illustrated as medians and box plots for each biomarker. Differences among patient groups for demographic and clinical characteristics were evaluated by Chi-square test. Spearman test correlation was applied for biomarker correlation for all patient groups. Biomarker levels were also compared among different groups using Kruskal-Wallis rank sum test when appropriate; when significant difference was obtained, the Wilcoxon nonparametric test was applied for pairwise group comparison.

All statistical tests were performed with “R” open source software. A p value less than 0.05 was considered as statistically significant.

### Results

TABLE 1
Baseline demographic and clinical characteristics of rule out (no
ACS), unstable angina and myocardial infarction (MI) individuals

| | Rule out | Unstable | | No ACS vs | | |
|---|---|---|---|---|---|---|
| | (no ACS) | angina | MI | unstable | no ACS vs | unstable angina |
| | Mean ± SD | Mean ± SD | Mean ± SD | angina | Ml | vs MI |
| | (Min-Max) | (Min-Max) | (Min-Max) | p | p | p |
| Mean age, yr | 49.68 ± 15.87 | 58.68 ± 16.79 | 79.14 ± 12.39 | 0.143 | 9⁻⁷ | 0.0007 |
| | (23-86) | (38-86) | (43-95) | | | |
| Sex, male/female (%) | 74.2/25.8 | 72.2/27.8 | 33.3/66.7 | 0.879 | 0.0034 | 0.015 |
| Current smokers, n | 32.3 | 44.4 | 4.7 | 0.109 | 0.014 | 0.00043 |
| (%) | | | | | | |
| Hypertension, n (%) | 22.6 | 44.5 | 61.9 | 0.109 | 0.0042 | 0.27 |
| Diabetes Mellitus, n | 9.7 | 5.5 | 28.6 | 0.611 | 0.08 | 0.06 |
| (%) | | | | | | |
| Hypercholesterolemia, | 19.3 | 22.2 | 4.7 | 0.809 | 0.129 | 0.104 |
| n (%) | | | | | | |
| Renal failure, n (%) | ND | ND | 19.5 | — | — | — |

Data are means ± Standard deviation.
ND: not documented

TABLE 2
Plasma biomarker levels in control, rule out (no Acute Coronary Syndrome),
unstable angina and myocardial infraction (MI) patients

| | Biomarker Levels | | | | | | | | | | | |
|---|---|---|---|---|---|---|---|---|---|---|---|---|
| | Control | | | Rule out (no ACS) | | | Unstable | | | MI | | |
| | Mean (SD) | | p | Mean (SD) | | p | Mean (SD) | | p | Mean (SD) | | p |
| CRP, mg/l | 1.32 | (1.29) | ** | 3.62 | (4.90) | ** | 3.22 | (4.56) | * | 63.32 | (64.36) | *** |
| IL-6, pg/ml | 2.07 | (1.64) | * | 4.46 | (4.82) | * | 2.89 | (2.87) | * | 54.42 | (51.18) | *** |
| BNP, pg/ml | 8.64 | (9.33) | *** | 21.72 | (17.79) | ** | 104.55 | (192.94) | ** | 812.26 | (839.10) | *** |
| proBNP, pg/ml | 52.03 | (61.45) | ** | 68.45 | (78.57) | * | 1143.89 | (1635.96) | * | 2150.50 | (2388.48) | ** |
| D-Dimer, ng/ml | 272.19 | (130.52) | * | 488.27 | (550.65) | * | 309.98 | (363.22) | * | 7480.54 | (14927.78) | *** |
| PAPP-A, ng/ml | 58.87 | (115.94) | * | 44.80 | (26.63) | * | 51.28 | (57.51) | — | 117.45 | (347.26) | — |
| MPO, ng/ml | 160.39 | (80.06) | * | 127.70 | (71.32) | * | 166.48 | (132.80) | * | 212.31 | (167.41) | ** |
| sCD40L, ng/ml | 0.79 | (1.48) | — | 0.78 | (1.36) | — | 0.71 | (0.79) | — | 3.65 | (8.76) | — |
| cTnl, ng/ml | 0.00 | (0.00) | *** | 0.00156 | (0.0045) | ** | 0.0068 | (0.0109) | ** | 4.35 | (6.37) | *** |

Data are means ± Standard deviation.
— no group difference,
* Significant vs. one other group,
** Significant vs. two other groups,
*** Significant vs. three other groups.

### Data Analysis on Demographic and Clinical Characteristics

As illustrated in Table 1, patients admitted with acute chest pain and not diagnosed for ACS are statistically comparable to proven unstable patients for age, gender, smoker status, diabetes and hypercholesterolemia. When comparing myocardial infarction group to suspected ACS population, a significant difference was reported for age, gender, smoker status; metabolic profile. Diabetes and hypercholesterolemia profiles are however statistically similar between myocardial infarction group and suspected ACS population (Table 1).

### Data Analysis on Single Marker Evaluation in Normal and Patient Groups

Using Kruskall-Wallis analysis for multiple group comparisons, a highly significant group difference was observed for a series of biomarkers, such as cTnI (p=2.2⁻¹⁶), D-Dimer (p=5.04⁻¹⁴), BNP (p=2.2⁻¹⁶), proBNP (p=0.003), IL-6 (p=7.8⁻¹⁶), CRP (p=2.2⁻⁶) and MPO (p=0.009). No significant difference, however, was noted between all groups for PAPP-A (p=0.12) and sCD40L (p=0.89) as single marker. Plasma cTnI (expressed in ng/ml) is significantly higher in myocardial infarction as compared to control, rule out and unstable patients (p=6.15-9 to 2.2-16), in total accordance with inclusion criteria defined for the clinical study. Even with the applied cTnI cut-off at 0.04 ng/ml, a significant difference between controls and rule out population (p=0.01) and between controls and unstable patients (p=5.5⁻⁵) was also recorded.

As illustrated in Table 2, CRP and BNP levels were significantly higher in myocardial infarction as compared to discharged patients (p=7.8⁻¹¹ and 4.8⁻¹² respectively), unstable patients (p=6.3⁻⁹ and 1.49-7 respectively) or control groups (p=5.85⁻¹⁵ and 4.35⁻¹⁵ respectively). Control CRP and BNP levels were significantly lower to rule out population (p=0.03 and 1.08⁻⁵ respectively). A clear linear relationship was obtained between BNP and its precursor proBNP in controls (r²=0.81, p=1.99⁻¹²) and patient groups, (r²=0.92, p=1.2⁻¹³) as evaluated by the multiple r-squared. Plasma levels of PAPP-A, MPO and sCD40L were comparable between control, rule out and unstable angina populations (Table 2). In myocardial infarction group, all 8 biomarkers are significantly highly elevated as compared to control and suspected ACS patients (p<0.001, Table 2), corresponding to a clear activation of pathophysiological components of myocardial damage and dysfunction, inflammation and plaque rupture. High risk for cardiac adverse events could be easily followed by plasma levels of CRP, IL-6, BNP, proBNP, D-Dimer, PAPP-A, MPO, sCD40L and cTnI (Table 2).

Based on Receiving-Operating-Characteristic (ROC) analysis for clinical status and as summarized in Tables 3, 4, 5 and 6, the area under the curve (AUC) for individual marker, such as BNP, proBNP, IL-6 and CRP, succeeded in discriminating patients diagnosed for myocardial infarction, or rule out population (no ACS) or unstable angina from healthy subjects. At admission, single marker evaluation, for example BNP, proBNP, sCD40L or IL-6 did not improve risk stratification in chest pain patients suggestive of an ACS with normal plasma cTnI, as it did not discriminate unstable angina with no ST-elevation patients from discharged patients with no ACS (Tables 4, 5 and 6).

Based on the ROC analysis, single marker evaluation including marker accounting for myocardial damage (cTnI) and dysfunction (BNP and proBNP), inflammation (IL-6, CRP, MPO, sCD40L) and plaque rupture (PAPP-A and D-Dimer) did not prove alone to be a valuable diagnostic and prognostic tool to help clinicians to identify patients with suspected ACS to be safely and rapidly discharged from the emergency department.

### Data Analysis on Multimarker Evaluation for Diagnosis and Prognosis

Multimarker analysis with mROC (multiple Receiving-Operating-Characteristic) approach improved significantly AUC when comparing controls to myocardial infarction, rule out and unstable angina patients (Tables 3, 5 and 6). The marker combination composed of IL-6, CRP and cTnI or IL-6 and CRP or, IL-6 and BNP has a predictive value for higher risk of cardiovascular events in unstable patients as reported by the higher sensitivity (94.7, 100 and 97.4% respectively) and specificity (100, 86.4 and 86.4% respectively) (Table 4). In the same application, the marker combination panels composed of proBNP, CRP and PAPP-A, or proBNP, MPO and PAPP-A, or proBNP, IL-6 and PAPP-A, as well as proBNP, D-Dimer and PAPP-A are also of interest (100%, 77.8%, 88.9%, and 77.8% of sensitivity respectively) (table 6). The current proposed multimarker evaluation gives the opportunity to improve ACS diagnosis at admission in the emergency department, by discriminating safely and rapidly unstable patients from myocardial infarction patients. In the same manner, other marker combination panels for example, BNP, D-Dimer and CRP (AUC of 0.739), BNP, IL-6 and CRP (AUC of 0.736), PAPP-A, MPO and proBNP (AUC of 0.919), PAPP-A, CRP and proBNP (AUC of 0.879) in patients with suspicious ACS could also help clinicians to safely diagnose patients with normal plasma cTnI levels and to discriminate no ACS patients and unstable patients with no ST-elevation (Table 7).

Interestingly, the discriminating power for acute coronary syndrome from rule out population was improved when combining a series of markers, for example BNP, proBNP, D-Dimer, IL-6, CRP, PAPP-A and sCD40L in patients with normal plasma cTnI levels (Table 7). The multimarker evaluation is characterized by a high sensitivity (100%) and specificity (90.9%) added to an elevated AUC of 0.98 (Table 7), corresponding to an improved predictive value for ACS diagnosis in patients with chest discomfort and normal plasma cTnI at emergency department admission.

Thus, the sensitivity and specificity of association of markers accounting for pathophysiological status such as myocardial damage (cTnI) and dysfunction (BNP and proBNP), inflammation (CRP, MPO, IL-6 and sCD40L) and plaque rupture (PAPP-A and D-Dimer) to discriminate patients with no ACS and discharged from the emergency department from diagnosed unstable angina are highly improved to 100% and 90.9% respectively (Table 7). This multimarker combination has an AUC of 0.98, corresponding to efficient screening of patients with chest pain at admission and to increase safely discharge of no ACS patients.

TABLE 3
Receiving-Operating-Characteristic analysis data for
single and multimarker combination for myocardial infarction
patients when compared to normal subjects:

| | | Sensitivity | Specificity | |
|---|---|---|---|---|
| Marker | Threshold | (%) | (%) | AUC |
| BNP, pg/ml | 37 | 97.4 | 97.9 | 0.997 |
| MPO, ng/ml | 173.8 | 52.6 | 72.3 | 0.595 |
| D-Dimer, ng/ml | 694.3 | 86.8 | 100 | 0.968 |
| CRP, mg/l | 3.42 | 100 | 95.7 | 0.995 |
| IL-6, pg/ml | 5 | 94.7 | 93.6 | 0.99 |
| BNP and CRP ₐ | 0.081 | 97.4 | 100 | 0.999 |
| BNP and IL-6 _{b} | 0.111 | 100 | 100 | 1 |
| BNP, IL-6 and CRP _{c} | 0.112 | 100 | 100 | 1 |

AUC means area under the curve. Curves are based on models of prediction of cardiovascular risk by calculating power of sensitivity (%) and specificity (%) for single marker and combined panel. The equations for the marker combinations calculated by the mROC program were:
ₐ Z = 0.001 BNP + 0.011 CRP,
_{b} Z = 0.001 BNP + 0.014 IL-6,
_{c} Z = 0.001 BNP + 0.01 IL-6 + 0.009 CRP.
Using these equations a new virtual marker (Z) was calculated.

TABLE 4
Receiving-Operating-Characteristic analysis data for
single and multimarker combination for myocardial infarction
patients when compared to unstable patients:

| | | Sensitivity | Specificity | |
|---|---|---|---|---|
| Marker | Threshold | (%) | (%) | AUC |
| Tnl, ng/ml | 0.05 | 86.8 | 100 | 0.949 |
| BNP, pg/ml | 206 | 84.2 | 86.4 | 0.91 |
| MPO, ng/ml | 116.9 | 78.9 | 59.1 | 0.659 |
| D-Dimer, ng/ml | 387.3 | 94.7 | 81.8 | 0.947 |
| CRP, mg/l | 4.26 | 94.7 | 81.8 | 0.953 |
| IL-6, pg/ml | 6 | 92.1 | 86.4 | 0.967 |
| PAPP-A, ng/ml | 37.2 | 63.2 | 59.1 | 0.607 |
| IL-6, CRP and Tnl ₐ | 0.321 | 94.7 | 100 | 0.993 |
| IL-6 and CRP _{b} | 0.13 | 100 | 86.4 | 0.978 |
| IL-6 and BNP _{c} | 0.124 | 97.4 | 86.4 | 0.967 |

AUC means area under the curve. Curves are based on models of prediction of cardiovascular risk by calculating power of sensitivity (%) and specificity (%) for single marker and combined panel. The equations for the marker combinations calculated by the mROC program were:
ₐ Z = 0.015 IL-6 + 0.008 CRP + 0.093 Tnl,
_{b} Z = 0.014 IL-6 + 0.01 CRP,
_{c} Z = 0.015 IL-6 + 0.001 BNP.
Using these equations a new virtual marker (Z) was calculated.

TABLE 5
Receiving-Operating-Characteristic analysis data for
single and multimarker combination for rule out (no
ACS) population when compared to normal subjects:

| | | Sensitivity | Specificity | |
|---|---|---|---|---|
| Marker | Threshold | (%) | (%) | AUC |
| BNP, pg/ml | 7 | 90.6 | 59.6 | 0.793 |
| proBNP, pg/ml | 37 | 54.5 | 58.3 | 0.529 |
| MPO, ng/ml | — | 59.4 | 68.1 | 0.64 |
| D-Dimer, ng/ml | 261.4 | 50 | 59.6 | 0.533 |
| CRP, mg/l | 1.72 | 56.3 | 74.5 | 0.641 |
| IL-6, pg/ml | 3 | 50 | 80.9 | 0.602 |
| PAPP-A, ng/ml | 38.7 | 72.7 | 69.4 | 0.672 |
| sCD40L, ng/ml | 0.36 | 90.9 | 61.1 | 0.718 |
| BNP, CRP and proBNP ₐ | 0.116 | 100 | 77.8 | 0.934 |
| BNP and proBNP _{b} | 0.126 | 100 | 77.8 | 0.932 |
| BNP and CRP _{c} | 0.37 | 84.4 | 68.1 | 0.81 |
| BNP, proBNP and PAPP-A _{d} | 0.007 | 100 | 77.8 | 0.932 |
| BNP, CRP and MPO ₑ | 0.016 | 78.1 | 78.7 | 0.83 |
| BNP, proBNP and sCD40L _{f} | 0.215 | 100 | 75 | 0.924 |
| BNP, IL-6, CRP and MPO _{g} | 0.112 | 78.1 | 74.5 | 0.825 |
| BNP, proBNP and IL-6 ₕ | 0.118 | 100 | 83.3 | 0.934 |
| BNP, proBNP and MPO ᵢ | 0.1 | 90.9 | 83.3 | 0.909 |
| proBNP, sCD40L and CRP ⱼ | 0.151 | 100 | 50 | 0.747 |
| proBNP, sCD40L and IL-6 ₖ | 0.23 | 72.7 | 55.6 | 0.639 |
| proBNP, sCD40L and MPO ₗ | 0.216 | 72.7 | 55.6 | 0.652 |
| proBNP and sCD40L ₘ | 0.107 | 90.9 | 52.8 | 0.672 |

AUC means area under the curve. Curves are based on models of prediction of cardiovascular risk by calculating power of sensitivity (%) and specificity (%) for single marker and combined panel. The equations for the marker combinations calculated by the mROC program were:
ₐ Z = 0.109 BNP − 0.019 CRP − 0.022 proBNP,
_{b} Z = 0.108 BNP − 0.022 proBNP,
_{c} Z = 0.031 BNP + 0.079 CRP,
_{d} Z = 0.109 BNP − 0.022 proBNP − 0.003 PAPP-A,
ₑ Z = 0.031 BNP + 0.102 CRP − 0.004 MPO,
_{f} Z = 0.107 BNP − 0.022 proBNP + 0.074 sCD40L,
_{g} Z = 0.031 BNP + 0.041 IL-6 + 0.078 CRP − 0.004 MPO,
ₕ Z = 0.115 BNP − 0.092 IL-6 − 0.023 proBNP,
ᵢ Z = −0.002 MPO + 0.110 BNP − 0.023 proBNP,
ⱼ Z = 0.156 sCD40L + 0.112 CRP − 0.001 proBNP,
ₖ Z = 0.12 sCD40L + 0.054 IL-6 + 0.001 proBNP,
ₗ Z = 0.107 sCD40L + 0.001 MPO + 0.001 proBNP,
ₘ Z = 0.105 sCD40L + 0.001 proBNP.
Using these equations a new virtual marker (Z) was calculated.

TABLE 6
Receiving-Operating-Characteristic analysis data for single
and multimarker combination for patients diagnosed for
unstable angina when compared to normal subjects:

| | | Sensitivity | Specificity | |
|---|---|---|---|---|
| Marker | Threshold | (%) | (%) | AUC |
| BNP, pg/ml | 10 | 72.7 | 70.2 | 0.787 |
| proBNP, pg/ml | 57 | 66.7 | 72.2 | 0.719 |
| MPO, ng/ml | 163.5 | 66.7 | 77.8 | 0.62 |
| CRP, mg/l | 1.9 | 45.5 | 76.6 | 0.588 |
| IL-6, pg/ml | 1.8 | 45.5 | 42.6 | 0.534 |
| sCD40L, ng/ml | 0.28 | 88.9 | 61.1 | 0.665 |
| BNP and CRP ₐ | 0.219 | 68.2 | 89.4 | 0.787 |
| proBNP, CRP and IL-6 _{b} | 0.922 | 55.6 | 100 | 0.787 |
| BNP, CRP and IL-6 _{c} | 0.132 | 63.6 | 91.5 | 0.802 |
| BNP, CRP and PAPP-A _{d} | 0.162 | 68.2 | 89.4 | 0.805 |
| proBNP, CRP and PAPP-A ₑ | 0.079 | 100 | 80.6 | 0.935 |
| proBNP, MPO and PAPP-A _{f} | 0.308 | 77.8 | 83.3 | 0.88 |
| proBNP, BNP and CRP _{g} | 0.504 | 66.7 | 94.4 | 0.744 |
| proBNP, D-Dimer and | 0.115 | 77.8 | 86.1 | 0.787 |
| PAPP-A ₕ | | | | |
| proBNP, IL-6 and PAPP-A ᵢ | 0.111 | 88.9 | 83.3 | 0.858 |
| proBNP, sCD40L and | 0.031 | 66.7 | 100 | 0.815 |
| PAPP-A ⱼ | | | | |
| proBNP and PAPP-A ₖ | 0.02 | 66.7 | 100 | 0.79 |

AUC means area under the curve. Curves are based on models of prediction of cardiovascular risk by calculating power of sensitivity (%) and specificity (%) for single marker and combined panel. The equations for the marker combinations calculated by the mROC program were:
ₐ Z = 0.002 BNP + 0.078 CRP,
_{b} Z = 0.001 proBNP + 0.133 CRP − 0.07 IL-6,
_{c} Z = 0.005 BNP + 0.137 CRP − 0.208 IL-6,
_{d} Z = 0.003 BNP + 0.077 CRP − 0.002 PAPP-A,
ₑ Z = −0.01 PAPP-A + 0.146 CRP + 0.001 proBNP,
_{f} Z = −0.01 PAPP-A + 0.003 MPO + 0.001 proBNP,
_{g} Z = −0.002 BNP + 0.130 CRP + 0.001 proBNP,
ₕ Z = −0.007 PAPP-A + 0.001 D-Dimer + 0.001 proBNP,
ᵢ Z = −0.008 PAPP-A + 0.111 IL-6 + 0.001 proBNP,
ⱼ Z = −0.006 PAPP-A + 0.021 sCD40L + 0.001 proBNP,
ₖ Z = −0.006 PAPP-A + 0.001 proBNP.
Using these equations a new virtual marker (Z) was calculated.

TABLE 7
Receiving-Operating-Characteristic analysis data for single
and multimarker combination for patients diagnosed for unstable
angina when compared to rule out population:

| | | Sensitivity | Specificity | |
|---|---|---|---|---|
| Marker | Threshold | (%) | (%) | AUC |
| BNP, pg/ml | 88 | 44.44 | 100 | 0.636 |
| MPO, ng/ml | 163.5 | 66.7 | 63.6 | 0.606 |
| IL-6, pg/ml | 1.7 | 77.8 | 63.6 | 0.687 |
| proBNP. pg/ml | 57 | 66.7 | 63.6 | 0.692 |
| BNP, D-Dimer and CRP ₐ | — | 72.7 | 59.4 | 0.739 |
| BNP, IL-6 and CRP _{b} | — | 72.7 | 56.3 | 0.736 |
| PAPP-A, MPO and proBNP _{c} | 0.296 | 77.8 | 100 | 0.919 |
| PAPP-A, CRP and proBNP _{d} | −0.252 | 88.9 | 90.9 | 0.879 |
| PAPP-A, sCD40L and | −0.061 | 66.7 | 100 | 0.808 |
| proBNP ₑ | | | | |
| PAPP-A, IL-6 and proBNP _{f} | −0.163 | 77.8 | 90.9 | 0.848 |
| PAPP-A, BNP and proBNP _{g} | −0.313 | 77.8 | 90.9 | 0.848 |
| PAPP-A and proBNP ₕ | −0.31 | 77.8 | 90.9 | 0.848 |
| IL-6, CRP and proBNP ᵢ | 0.342 | 55.6 | 90.9 | 0.707 |
| BNP, proBNP, D-Dimer, | 0.38 | 100 | 90.9 | 0.98 |
| IL-6, CRP, PAPP-A, | | | | |
| sCD40L, MPO ⱼ | | | | |

AUC means area under the curve. Curves are based on models of prediction of cardiovascular risk by calculating power of sensitivity (%) and specificity (%) for single marker and combined panel. The equations for the marker combinations calculated by the mROC program were:
ₐ Z = 0.003 BNP − 0.001 D-Dimer − 0.007 CRP,
_{b} Z = 0.004 BNP + 0.041 CRP − 0.127 IL-6,
_{c} Z = −0.027 PAPP-A + 0.005 MPO + 0.002 proBNP,
_{d} Z = −0.03 PAPP-A + 0.155 CRP + 0.002 proBNP,
ₑ Z = −0.016 PAPP-A − 0.156 sCD40L + 0.001 proBNP,
_{f} Z = −0.016 PAPP-A − 0.034 IL-6 + 0.001 proBNP,
_{g} Z = −0.016 PAPP-A − 0.001 BNP + 0.001 proBNP,
ₕ Z = −0.015 PAPP-A + 0.001 proBNP,
ᵢ Z = −0.06 IL-6 + 0.057 CRP + 0.001 proBNP
ⱼ Z = 0.016 MPO − 0.068 PAPP-A − 0.002 D-Dimer + 0.011 BNP − 0.721 sCD40L − 0.131 IL-6 + 0.249 CRP + 0.002 proBNP.
Using these equations a new virtual marker (Z) was calculated.

All bibliographic references cited herein are incorporated by reference.

1. A method for the prognosis of a vascular event in a patient suspected of being at risk for a vascular event, said patient presenting:
no elevation of the ST segment as seen on an electrocardiogram, and/or
a normal level of at least one necrosis marker,
wherein the presence and/or levels of at least two different biochemical markers are measured in a biological sample of said patient, said biochemical markers being selected from the group consisting of the following classes of markers:
atherosclerotic plaque unstability markers,
atherosclerotic plaque disruption markers,
coronary inflammation markers,
pre-thrombotic status markers,
thrombosis markers,
ischemic markers,
necrosis markers, and
myocardial dysfunction markers,
whereby the probability that the patient will experience a vascular event is deduced from the measured presence and/or levels of the biochemical markers.

2. The method for the prognosis of a vascular event according to claim 1, wherein, the patient presents a chest pain.

3. The method for the prognosis of a vascular event according to claim 1, wherein the patient presents a normal cardiac Troponin level and/or a normal CK level.

4. The method for the prognosis of a vascular event according to claim 1, wherein the patient presents a normal cardiac Troponin I or cardiac Troponin T level and no elevation of the ST segment.

5. The method for the prognosis of a vascular event according to claim 1, wherein:
the atherosclerotic plaque unstability marker is selected from the group consisting of MPO, sCD40L, IL-6, PAPP-A and choline;
the atherosclerotic plaque disruption marker is selected from the group consisting of MPO, sCD40L, MMPs, Choline and PAPP-A;
the coronary inflammation marker is selected from the group consisting of MPO, sCD40L, CRP and IL-6;
the pre-thrombotic status marker is sCD40L or fibrinogen;
the thrombosis marker is selected from the group consisting of D-Dimers;
the ischemic marker is selected from the group consisting of IMA, cTnI, cTnT, and FABP;
the necrosis marker is selected from the group consisting of cTnI, cTnT, CK, myoglobin and FABP;
the myocardial dysfunction marker is selected from the group consisting of BNP, NT-proBNP and proBNP.

6. The method for the prognosis of a vascular event according to claim 1, wherein the biochemical markers are selected from the group consisting of the following combinations:
proBNP and PAPP-A, and optionally a marker selected from the group consisting of CRP, IL-6, MPO, sCD40L, D-Dimer, and BNP;
proBNP, IL-6, and a marker selected from the group consisting of CRP, sCD40L, and BNP;
proBNP and BNP, and optionally a marker selected from the group consisting of CRP, sCD40L, and MPO;
BNP or proBNP, and CRP, and optionally a marker selected from the group consisting of IL-6, MPO, PAPP-A, and D-Dimer;
proBNP or BNP, and sCD40L, and optionally a marker selected from the group consisting of CRP and MPO;
CRP and IL-6;
BNP or proBNP, and IL-6, and optionally CRP and MPO;
proBNP, PAPP-A, CRP, BNP, D-Dimer, IL-6, sCD40L, and MPO.

7. The method for the prognosis of a vascular event according to claim 1, wherein the vascular event is selected from the group consisting of myocardial vascular-related death, myocardial infarction, congestive-heart failure, vascular-related hospitalization, and revascularization procedure.

8. A method for diagnosing an acute coronary syndrome (ACS) in a patient presenting:
no elevation of the ST segment as seen on an electrocardiogram, and/or
a normal level of at least one necrosis marker,
comprising measuring the presence and/or levels of at least two different biochemical markers in a biological sample of said patient, said biochemical markers being selected from the group consisting of the following classes of markers:
atherosclerotic plaque unstability markers,
atherosclerotic plaque disruption markers,
coronary inflammation markers,
pre-thrombotic status markers,
thrombosis markers,
ischemic markers,
necrosis markers, and
myocardial dysfunction markers,
whereby it is determined if said patient suffers from ACS.

9. The method for diagnosing an ACS according to claim 8, wherein the patient presents a chest pain.

10. A method for diagnosing an ACS according to claim 8, wherein the patient presents a normal cardiac Troponin level and/or a normal CK level.

11. The method for diagnosing an ACS according to claim 8, wherein the patient presents a normal cardiac Troponin I or cardiac Troponin T level and no elevation of the ST segment.

12. A method for diagnosing ACS according to claim 8, wherein the biochemical markers are selected from the group consisting of the following combinations of markers:
proBNP and PAPP-A, and optionally a marker selected from the group consisting of CRP, IL-6, MPO, sCD40L, D-Dimer, and BNP;
proBNP, IL-6, and a marker selected from the group consisting of CRP, sCD40L, and BNP;
proBNP and BNP, and optionally a marker selected from the group consisting of CRP, sCD40L, and MPO;
BNP or proBNP, and CRP, and optionally a marker selected from the group consisting of IL-6, MPO, PAPP-A, and D-Dimer;
proBNP or BNP, and sCD40L, and optionally a marker selected from the group consisting of CRP and MPO;
CRP and IL-6;
BNP or proBNP, and IL-6, and optionally CRP and MPO;
proBNP, PAPP-A, CRP, BNP, D-Dimer, IL-6, sCD40L, and MPO;

13. A method for determining the condition of an individual suspected of having an ACS comprising measuring the presence and/or levels of at least two different biochemical markers in a biological sample of said patient, said biochemical markers being selected from the group consisting of the following combinations of markers:
proBNP and PAPP-A, and optionally a marker selected from the group consisting of CRP, IL-6, MPO, sCD40L, D-Dimer, and BNP;
proBNP, IL-6, and a marker selected from the group consisting of CRP, sCD40L, and BNP;
proBNP and BNP, and optionally a marker selected from the group consisting of CRP, sCD40L, and MPO;
BNP or proBNP, and CRP, and optionally a marker selected from the group consisting of IL-6, MPO, PAPP-A, and D-Dimer;
proBNP or BNP, and sCD40L, and optionally a marker selected from the group consisting of CRP and MPO;
CRP and IL-6;
BNP or proBNP, and IL-6, and optionally CRP and MPO;
proBNP, PAPP-A, CRP, BNP, D-Dimer, IL-6, sCD40L, and MPO;
whereby the condition of the individual is determined.

14. The method for determining the condition of an individual suspected of having an ACS according to claim 13, wherein the condition is selected from the group constituted of:
no ACS, and
unstable angina,

15. The method for determining the condition of an individual suspected of having an ACS according to claim 13, wherein (i) the level of at least one necrosis marker of the individual and (ii) the presence or absence of an elevation of the ST segment as seen on an electrocardiogram of the patient are determined.

16. The method for determining the condition of an individual suspected of having an ACS according to claim 15, wherein the necrosis marker is cardiac Troponin I or cardiac Troponin T.

17. A kit intended for the prognosis of a vascular event in a patient, for diagnosing an ACS, or for determining the condition of an individual suspected of having an ACS, said kit comprising ligands specific respectively to the markers of the combinations of markers selected from the group constituted of:
proBNP and PAPP-A, and optionally a marker selected from the group consisting of CRP, IL-6, MPO, sCD40L, D-Dimer, and BNP;
proBNP, IL-6, and a marker selected from the group consisting of CRP, sCD40L, and BNP;
proBNP and BNP, and optionally a marker selected from the group consisting of CRP, sCD40L, and MPO;
BNP or proBNP, and CRP, and optionally a marker selected from the group consisting of IL-6, MPO, PAPP-A, and D-Dimer;
proBNP or BNP, and sCD40L, and optionally a marker selected from the group consisting of CRP and MPO;
CRP and IL-6;
BNP or proBNP, and IL-6, and optionally CRP and MPO;
proBNP, PAPP-A, CRP, BNP, D-Dimer, IL-6, sCD40L, and MPO.

18. The kit according to claim 17, comprising one specific ligand to cardiac Troponin I or cardiac Troponin T.

19. The kit according to claim 17, wherein the specific ligands are selected from the group constituted of:
polyclonal, monoclonal and recombinant antibodies, or fragments thereof,
phage antibodies (PhAbs),
llama and camel antibodies,
aptamers.
